(19) United States
(12) Patent Application Publication
(10) Pub. No.: US 2010/0331944 A1
(43) Pub. Date: Dec. 30, 2010

(54) ELECTRODE ARRANGEMENT WITH A STIMULATION ELECTRODE FOR THE LEFT VENTRICLE

(76) Inventor: Peter Osypka, Rheinfelden-Herten (DE)

Correspondence Address: VOLPE AND KOENIG, P.C., UNITED PLAZA, 30 SOUTH 17TH STREET, PHILADELPHIA, PA, 19103, US

(21) Appl. No.: 12/819,447

(22) Filed: Jun. 21, 2010

(30) Foreign Application Priority Data
Jun. 25, 2009 (DE) 102009030340.5-54

(51) Int. Cl. A61N 1/05

(52) U.S. Cl. 607/127; 607/122

(57) ABSTRACT

An electrode arrangement has a stimulation electrode (7) that can be implanted transvenously for the Tawara node (5) allocated to the left ventricle (3) of a human heart (1). In order to not have to feed the stimulation electrode (7) via coronary veins, an element that can be inserted into the right ventricle (2) and that is used for piercing or forming a channel in the tissue of the heart (1) toward the Tawara node (5) of the left ventricle (3) is part of the electrode arrangement, so that the stimulation electrode (7) can be implanted in the region of the Tawara node (5) of the left ventricle (3). The feeding element can be a hollow needle (8) or a first stimulation electrode (6) for the right ventricle (2). The channel can be formed by a stiletto blade (9) or guide wire (10) or the helical coil of the second stimulation electrode (7) itself.

### CROSS-REFERENCE TO RELATED APPLICATIONS

This application claims the benefit of German Patent Application 10 2009 030 340.5-54, filed Jun. 25, 2009, which is incorporated herein by reference as if fully set forth.

### BACKGROUND

The invention relates to an electrode arrangement with a stimulation electrode that can be implanted transvenously for the Tawara node allocated to the left ventricle of a human heart, wherein the electrode arrangement has a means that can be inserted transvenously into the right ventricle for piercing or forming a channel in the tissue of the heart toward the Tawara node of the left ventricle and has a stimulation electrode that can be fed transvenously and into and through the right ventricle, as well as through the previously formed channel.

When the heart is especially weak, it is known to stimulate not only the right ventricle, but also, in many cases, the left ventricle of a heart. Up to now, however, there had been no accessibility for a stimulation electrode at the left ventricle and this was also not useful due to the high blood pressure prevailing there. Thus, up to now aid was given in that a stimulation electrode was brought into the vicinity of the Tawara node allocated to the left ventricle and placed there via the coronary sinus and the veins located on the left ventricle on the outside. Here, this stimulation electrode must be inserted against the venous blood flow and it is difficult to intersect the most favorable outer vein that has branches.

From US 2006/0224224 A1, an electrode arrangement of the type noted above is known in which a stimulation electrode is installed in the right ventricle and another electrode via the coronary sinus (FIG. 1) or from the right ventricle into the cardiac septum to the left ventricle, that is, into the septum (FIG. 5). In both cases, two stimulation electrodes are implanted one next to the other, which is to be considered difficult, because each of the two stimulation electrodes must be installed individually, but in a targeted manner.

### SUMMARY

Thus the object arises of creating an electrode arrangement of the type defined above that simplifies, in particular, the placement of the stimulation electrode used for stimulating the left ventricle.

To meet this objective, it is provided that the electrode arrangement with a means that can be inserted transvenously into the right ventricle for piercing or for forming a channel in the tissue of the heart toward the Tawara node of the left ventricle and with a stimulation electrode that can be fed transvenously and in and through the right ventricle, as well as through the previously formed channel, for the left ventricle has a feed device, namely a first stimulation electrode for stimulating the right ventricle with a lumen arranged in its interior and it is provided that the second stimulation electrode provided for the left ventricle can be inserted through the lumen of this first stimulation electrode.

In this way it is possible to feed a stimulation electrode for the Tawara node of the left ventricle through the right ventricle, which functions similar to the feeding of a stimulation electrode in the right ventricle and which avoids indirect paths via coronary veins. In this way it is further possible to look for an optimal or especially favorable stimulation position instead of being dependent on the random arrangement of the coronary veins.

Through the use of a first stimulation electrode for the right ventricle as a feed device for the second stimulation electrode, the patient can be provided with two stimulation electrodes without one of these stimulation electrodes having to be fed via the coronary veins. Instead, for the case of the first stimulation electrode for the right ventricle, its lumen can be used to move the second stimulation electrode through this lumen and through the right ventricle to its designated position.

Here it is favorable when the second stimulation electrode for the left ventricle or its Tawara node has an inner, continuous lumen in which a stiletto blade or a guide wire fits and it is favorable when the stimulation electrode can be moved into its position of use by a previously installed guide wire or a previously implanted stiletto blade. For example, initially a stiletto blade could also be used to form a channel from the right ventricle to the Tawara node of the left ventricle, after which the stimulation electrode can be moved via this channel, and indeed simultaneously with a first stimulation electrode for the right ventricle that initially holds it.

The second stimulation electrode can be a coil electrode that has, on its distal end, a helical coil that forms the cathode. This stimulation electrode can be anchored correspondingly well in its position of use by a screwing-in process.

In this way, a construction is possible to the extent that the helical coil provided on the distal end on the stimulation electrode for the left ventricle is shaped as a drill bit and is used itself to form the channel for the placement of the stimulation electrode for the left ventricle. The stimulation electrode can initially be fed via the right ventricle and can then be drilled and placed with its helical coil used as a drill bit from there into the tissue of the Tawara node belonging to the left ventricle.

The electrode arrangement according to the invention provides, in an especially favorable way, that a first stimulation electrode to be placed in the right ventricle has an inner channel or a lumen that opens on the distal end, that the second stimulation electrode for the left ventricle can move freely and is arranged displaceable in this channel or lumen, with the distal operational end of this stimulation electrode projecting past the distal end of the first stimulation electrode in the position of use, that the element for penetrating the heart tissue is provided on the distal end of the first stimulation electrode in the position of use, in order to create the channel from the right ventricle to the Tawara tissue of the left ventricle, and that the displaceable second stimulation electrode is displaceable with its distal end into and through this channel into its position of use on or close to the Tawara node of the left ventricle. This produces a compact and easy-to-handle electrode arrangement that comprises two stimulation electrodes, namely one for the right and one for the left ventricle and that can be implanted in a way that is typical for stimulation electrodes for the right ventricle. The channel from the right ventricle outward to the tissue of the Tawara node of the left ventricle is formed only after the first implantation step and the second stimulation electrode that has already been carried along is shifted into its final position of use partially from the first stimulation electrode.

Through the first stimulation electrode that can be placed in the right ventricle, in this way an element for forming a channel in the heart tissue and/or a stiletto blade and/or a guide wire can be displaceable and by this the second stimulation electrode can be placed in the tissue of the left ventricle.

It is favorable when each of the two stimulation electrodes has a bipolar construction and when both stimulation electrodes have, on each of their distal ends, the cathode and the corresponding anode at a distance to the cathode in the direction toward the proximal end. This allows the desired bipolar stimulation both in the right and also in the left ventricle.

Primarily through the combination of individual or multiple features and measures described above, an electrode arrangement is produced that allows the placement of a stimulation electrode in the region of the Tawara node belonging to the left ventricle without having to perform unreliable feeding via the coronary veins. Instead, this stimulation electrode can be implanted via the right ventricle, so that this implanting procedure is similar at least to the implantation that has already been typical for a long time of a stimulation electrode in the right ventricle. In this way, advantageously the first stimulation electrode for the right ventricle is used as an implantation aid for the second stimulation electrode, so that the first stimulation electrode has an additional function.

### BRIEF DESCRIPTION OF THE DRAWINGS

Below, an embodiment of the invention is described in detail with reference to the drawing. Shown in a partially schematized views are:

FIG. 1 is a section diagram of a human heart with the Tawara nodes between the right and the left ventricle and with part of the electrode arrangement according to the invention, namely with a first stimulation electrode that is implanted in the right ventricle, and

FIG. 2 is a view of the arrangement according to the invention in which the first stimulation electrode for the right ventricle is installed according to FIG. 1 and is used as a feeding device for a second stimulation electrode that is already placed and implanted in its position of use in the heart tissue close to the Tawara node of the left ventricle and has created itself, with its helical coil, the channel in the heart tissue for its placement.

### DETAILED DESCRIPTION OF THE PREFERRED EMBODIMENTS

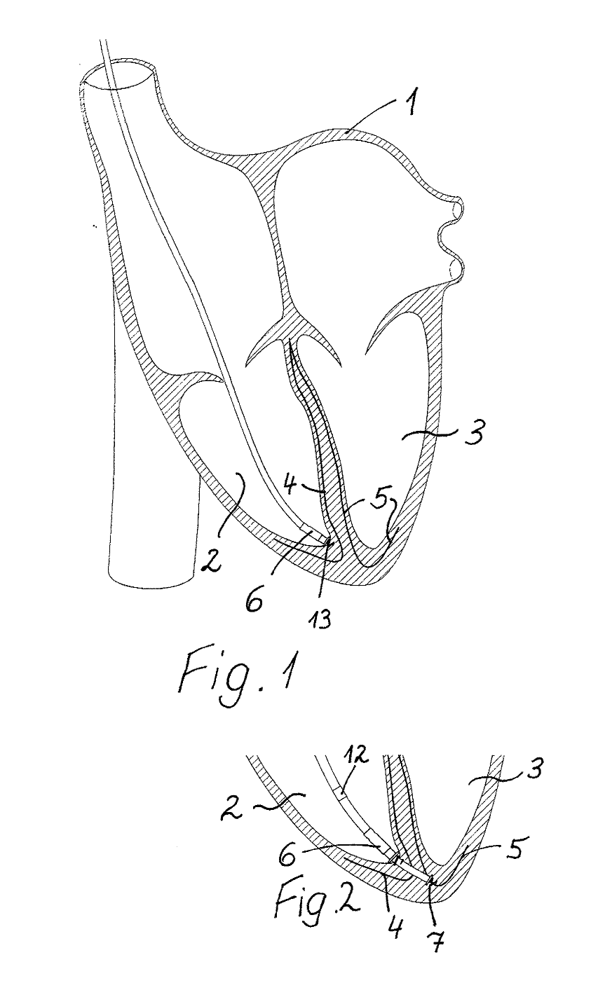

In FIG. 1, a vertical longitudinal section through a human heart 1 is shown schematically, wherein the heart has a right ventricle 2, a left ventricle 3, and Tawara nodes 4 and 5 associated with these ventricles. The Tawara node 5 is associated with the left ventricle 3.

FIG. 1 also shows a first stimulation electrode 6 for the right ventricle 2 that is implanted transvenously. This is part of an electrode arrangement still to be described, for example, according to FIG. 2.

In FIG. 2, an electrode arrangement with a stimulation electrode 7 that can be implanted transvenously for the Tawara node 5 allocated to the left ventricle 3 of the heart 1 is shown. A feeding device belongs to this electrode arrangement.

For this purpose, the first stimulation electrode 6 to be placed in the right ventricle 2 has an inner channel or a lumen that opens freely on the distal end, so that this stimulation electrode 6 can be used as a feeding device or feeding means for the second stimulation electrode 7. In this channel or lumen, the second stimulation electrode 7 for the left ventricle 3 is arranged so that it can move or be displaced, namely, freely.

In the position of use shown in FIG. 2, the second stimulation electrode 7 projects beyond the distal end of the first stimulation electrode 6, that is, it is shifted a corresponding distance. Here, a corresponding opening or a channel through which the stimulation electrode 7 for the left ventricle 5 can be fed is formed with an element that can be inserted into the right ventricle 2 for piercing or forming a channel in the tissue of the heart toward the Tawara node 5 of the left ventricle 3.

Here, in the embodiment, the stimulation electrode 7 for the left ventricle 5 is likewise a coil electrode that has a helical coil shaped as a drill bit on its distal end, so that it itself is used to form the channel or the opening for the placement of this stimulation electrode 7. Indeed, initially a stiletto blade or guide wire could be inserted, but the second stimulation electrode 7 itself is or includes the element for forming the corresponding channel opening for this stimulation electrode 7.

In a way that is not shown in detail, in the embodiment of the electrode arrangement according to the invention it is provided that each of the two stimulation electrodes 6 and 7 has a bipolar construction. Both stimulation electrodes 6 and 7 have, accordingly, on their distal ends, the cathode and the associated anode 12 is set at a distance to the cathode in the direction toward the proximal end. The cathode itself can be the helical coil that can be seen in the embodiment for both stimulation electrodes 6 and 7 or it can be closely adjacent to these electrodes.

The electrode arrangement has a stimulation electrode 7 that can be implanted transvenously for the Tawara node 5 allocated to the left ventricle 3 of a human heart 1. In order to not have to feed this stimulation electrode 7 via coronary veins, an element that can be inserted transvenously into the right ventricle 2 and that is used for piercing or forming a channel in the tissue of the heart 1 toward the Tawara node 5 of the left ventricle 3 belongs to the electrode arrangement, wherein the stimulation electrode 7 can be implanted in the region of the Tawara node 5 of the left ventricle 3. The feeding element can be a first stimulation electrode 6 for the right ventricle 2. The channel can be formed by the helical coil of the second stimulation electrode 7 itself.

1. An electrode arrangement that is adapted to be implanted transvenously for a Tawara node (5) associated with a left ventricle (3) of a human heart (1), the electrode arrangement comprising an element that is adapted to be inserted transvenously into the right ventricle (2) and that is used for piercing or forming a channel in tissue of the heart (1) toward the Tawara node (5) of the left ventricle (3) and a stimulation electrode (7) that can be fed transvenously and into and through the right ventricle, as well as through the previously formed channel, and a feeding device that comprises a first stimulation electrode (6) with a lumen arranged in its interior that is adapted to stimulate the right ventricle (2), and the second stimulation electrode (7) provided for the left ventricle (3) is insertable through the lumen of the first stimulation electrode (6).

2. The electrode arrangement according to claim 1, wherein the second stimulation electrode (7) for the Tawara node of the left ventricle (3) has an inner, continuous lumen in which a stiletto blade (9) or a guide wire (10) fits.

3. The electrode arrangement according to claim 1, wherein the second stimulation electrode (7) is a coil electrode that has, on a distal end, a helical coil that forms the cathode.

4. The electrode arrangement according to claim 3, wherein the helical coil provided on the second stimulation electrode on the distal end is constructed as a drill bit and is used as the element to form the channel for the placement of the second stimulation electrode (7).

5. The electrode arrangement according to claim 1, wherein the element that is adapted to form the channel in the heart tissue, a stiletto blade (9) or a guide wire (10) are shiftable through the first stimulation electrode (6) that is adapted to be placed in the right ventricle (2) to allow placement of the second stimulation electrode (7) in the tissue of the left ventricle (3).

6. The electrode arrangement according to claim 1, wherein each of the two stimulation electrodes has a bipolar construction and both of the stimulation electrodes have, on their distal ends, a cathode and a corresponding anode (12) that is set at a distance to the cathode in a direction toward a proximal end.
